(19) United States
(12) Patent Application Publication
(10) Pub. No.: US 2025/0195356 A1
(43) Pub. Date: Jun. 19, 2025

(54) MULTI-FUNCTION ALCOHOL-FREE HAIR CARE COMPOSITION

(71) Applicant: The Procter & Gamble Company, Cincinnati, OH (US)

(72) Inventors: SUPRIYA PUNYANI, NEW DELHI (IN); ZHE WEI TEO, SINGAPORE (SG)

(21) Appl. No.: 18/541,480

(22) Filed: Dec. 15, 2023

Related U.S. Application Data
(60) Provisional application No. 63/433,128, filed on Dec. 16, 2022.

(51) Int. Cl. A61K 8/49; A61K 8/34; A61K 8/365; A61K 8/42; A61K 8/67; A61K 8/73; A61K 8/9789; A61Q 5/02

(52) U.S. Cl. CPC A61K 8/4926; A61K 8/34; A61K 8/345; A61K 8/365; A61K 8/42; A61K 8/4993; A61K 8/675; A61K 8/73; A61K 8/9789; A61Q 5/02; A61K 2800/262; A61K 2800/30; A61K 2800/33

(57) ABSTRACT

The present invention is directed to a transparent hair care composition comprising a substituted 2-pyridinol-N-oxide material; a glycol; a non-ionic surfactant; a monocarboxylic acid or polyprotic acid; water or an aqueous carrier; wherein the composition has a pH from about 3.5 to about 6.0; and wherein the composition is free of alcohol.

### FIELD OF THE INVENTION

The present invention relates to a multi-function alcohol-free hair care composition.

### BACKGROUND OF THE INVENTION

For years, anti-dandruff rinse-offs (shampoo, scalp treatment) have been widely used to treat dandruff and to clean hair and scalp. However, for consumers who have severe dandruff issues, such as flaking, itching, or an oily or dry scalp, using only anti-dandruff shampoo is often not enough. This is because the deposition, or delivery of anti-dandruff active to the scalp is limited by use of just a single product. While there have been anti-dandruff conditioners in the market for many years, these rinse-off anti-dandruff conditioners are highly viscous and greasy-looking products. In fact, these products are often just regular hair conditioning products with anti-dandruff actives added in. As a result, most consumers apply these products carefully to their hair, while intentionally avoiding the products from touching their scalps, meaning the consumers do not receive the anti-dandruff benefit of the products. The leave-on anti-dandruff products are in various forms, such as spray tonics, lotions, and creams, but the problem with these products is that, while they may deliver an anti-dandruff benefit, all use ethanol to dissolve oil soluble anti-dandruff active, opaque, and sticky after application. Due to strong smell of ethanol and opaqueness of product, greasy in application it connotes full of chemicals and not desirable to use every day and these products are less popular in the market. Most of consumer, are looking for natural looking products which are transparent, ethanol-free, can provide scalp benefit such as anti-dandruff, anti-itch etc. and non-sticky after application. Therefore, there is a need for hair care composition that are multi-functional, that is, that consumers can apply to their scalp delivering scalp benefit such as anti-dandruff, anti-itch efficacy while transparent, ethanol-free and non-sticky after application that consumers expect and want.

In addition, consumers desire a minimal number of ingredients in their products and want to understand why each ingredient is needed. Many consumers look for labels stating the compositions are free of unnecessary chemicals, particularly for products that are applied directly to the scalp, which may already be sensitive due to the flaking and itching symptoms of dandruff. It follows then that the present invention has eliminated or minimized the number of ingredients that are not primarily functional to the consumer benefits, for example, preservatives, stabilizers, or solvents.

Therefore, there is a continuing need for leave-on that offer not only scalp benefit such as anti-dandruff, anti-itch benefits, but is ethanol-free, transparent, non-sticky after application, all while being formulated with natural and multi-functional materials.

### SUMMARY OF THE INVENTION

The present invention is directed to a transparent hair care composition comprising a substituted 2-pyridinol-N-oxide material; a glycol; a non-ionic surfactant; a monocarboxylic acid or polyprotic acid; water or an aqueous carrier; wherein the composition has a pH from about 3.5 to about 6.0; and wherein the composition is free of alcohol.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”. As used herein, “consisting essentially of” means that the composition or component may include additional ingredients, but only if the additional ingredients do not materially alter the basic and novel characteristics of the claimed compositions or methods.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials. All measurements are understood to be made at ambient conditions, where “ambient conditions” means conditions at about 25° C., under about one atmosphere of pressure, and at about 50% relative humidity (RH), unless otherwise designated. All numeric ranges are inclusive of narrower ranges; delineated upper and lower range limits are combinable to create further ranges not explicitly delineated.

Herein, “mixtures” is meant to include a simple combination of materials and any compounds that may result from their combination.

The term “molecular weight” or “M.Wt.” as used herein refers to the weight average molecular weight unless otherwise stated. “QS” means sufficient quantity for 100%.

“Apply” or “application” as used in reference to a composition, means to apply or spread the compositions of the present invention onto keratinous tissue such as the hair.

“Dermatologically acceptable” means that the compositions or components described are suitable for use in contact with human skin tissue without undue toxicity, incompatibility, instability, allergic response, and the like.

“Safe and effective amount” means an amount of a compound or composition sufficient to significantly induce a positive benefit.

“Rinse-off”' in reference to compositions, means compositions intended to be applied to keratinous substrate and subsequently removed by washing, rinsing or wiping within a few minutes or less from the application. These “rinse-off” compositions are to be distinguished from “leave-on” compositions, which are intended to be applied to and allowed to remain longer on the keratinous tissue.

“Leave-on,” in reference to compositions, means compositions intended to be applied to and allowed to remain on the keratinous tissue. These leave-on compositions are to be distinguished from compositions, which are applied to the hair and subsequently (in a few minutes or less) removed either by washing, rinsing, wiping, or the like. Leave-on compositions exclude rinse-off applications such as shampoos, rinse-off conditioners, facial cleansers, hand cleansers, body wash, or body cleansers. The leave-on compositions may be substantially free of cleansing or detersive surfactants. For example, “leave-on compositions” may be left on the keratinous tissue for at least 5 minutes. For example, leave-on compositions may comprise less than 1% detersive surfactants, less than 0.5% detersive surfactants, or 0% detersive surfactants. The compositions may, however, contain emulsifying, dispersing or other processing surfactants that are not intended to provide any significant cleansing benefits when applied topically to the hair.

“Soluble” means at least about 0.1 g of solute dissolves in 100 ml of solvent, at 25° C. and 1 atm of pressure.

“Hair,” as used herein, means mammalian hair including scalp hair, facial hair and body hair, particularly on hair on the human head and scalp.

“Cosmetically acceptable,” as used herein, means that the compositions, formulations or components described are suitable for use in contact with human keratinous tissue without undue toxicity, incompatibility, instability, allergic response, and the like. All compositions described herein which have the purpose of being directly applied to keratinous tissue are limited to those being cosmetically acceptable. “Derivatives,” as used herein, includes but is not limited to, amide, ether, ester, amino, carboxyl, acetyl, acid, salt and/or alcohol derivatives of a given compound.

“Polymer,” as used herein, means a chemical formed from the polymerisation of two or more monomers. The term “polymer” as used herein shall include all materials made by the polymerisation of monomers as well as natural polymers. Polymers made from only one type of monomer are called homopolymers. Polymers made from two or more different types of monomers are called copolymers. The distribution of the different monomers can be calculated statistically or block-wise-both possibilities are suitable for the present invention. Except if stated otherwise, the term “polymer” used herein includes any type of polymer including homopolymers and copolymers.

### Hair Care Composition

The present inventive hair care compositions provide a combination of multi-functional ingredients that can deliver multiple benefits, including anti-dandruff, anti-itch, soothing, fragrance, preservation, non-sticky feel and dry conditioning, all while having nine or fewer ingredients. The product appearance is transparent and spray form, however can be made between a cream and a lotion, and the appearance may be slight translucent. As the present invention has set out to solve the problem of providing hair care compositions that delivered the numerous benefits described while not having very many ingredients, it is further realized that single ingredients can serve multiple functions. The present invention is directed to formulate compositions with consumer-preferred natural ingredients. For example, the non-ionic surfactant polyethoxylated sorbitan, is naturally derived from vegetable oils and fats, which allows a formulator freedom to choose as emulsifier in non-alcohol containing chassis.

### Anti-Dandruff Active

Piroctone and salts of piroctone, such as piroctone olamine, are known to provide anti-dandruff benefit. However, piroctone olamine is often supplied as a crystal powder, and is also known to have very limited solubility in water, meaning the challenge is to assure solubilization of an anti-dandruff active in as simple as possible a material combination.

It has been found by the present invention that, certain solid organic compounds, such as piroctone and/or salts thereof, tend to crystallize in the following conditions:
when the amount of the solid organic compounds increases in the composition, especially 0.2% or above; and/or
when the composition has pH of from about 3.5 to about 6.

Piroctone olamine in a leave-on is usually solubilized by alcohol (e.g., ethanol) or emollient oil such as such triethylhexanoine, octyldodecyl myristate at high concentration (>30%). The oil is typically emulsified by suspending polymers (e.g., acrylates), resulting in an opaque product and highly viscous resulting in sticky feel after application. In contrast, the present invention does not use emollient oil, ethanol and tacky polymers, and instead uses a mixture of glycol and non-ionic surfactant, in a specific ratio to piroctone olamine, to get to a desired pH range for the piroctone olamine to function and deposit better on the scalp. This leads to anti-dandruff efficacy and non-sticky hair feel, while not using too much of the glycols.

In the present invention, the anti-dandruff active may be a substituted 2-pyridinol-N-oxide material, such as piroctone olamine or ciclopirox olamine. Piroctone olamine and ciclopirox olamine are the salt of piroctone and ciclopirox, respectively, with monoethanolamine. In the compositions having an acidic pH, these become protonated to become piroctone and ciclopirox. respectively. In the present invention, the composition may have an acidic pH, may have a pH from about 2 to about 6, may have a pH from about 3.5 to about 6, or may have a pH from about 10) 3.5 to about 5.

The present invention may also include one or more soluble scalp health actives, which may be one material or a mixture selected from the groups consisting of: azoles, such as climbazole, ketoconazole, itraconazole, econazole, and elubiol; hydroxy pyridones, such as piroctone olamine, ciclopirox, rilopirox, and MEA-Hydroxyoctyloxypyridinone; strobilurins such as azoxystrobin and metal chelators such as 1.10-phenanthroline.

The azole soluble scalp health active may be an imidazole selected from the group consisting of: benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof, or the azole anti-microbials is a triazole selected from the group consisting of: terconazole, itraconazole, and mixtures thereof. The azole soluble scalp health active may be ketoconazole. The sole soluble scalp health active may be ketoconazole.

These anti-dandruff and scalp health active compounds are not soluble in a typical solvent such as water or an aqueous mixture such as water containing even a high level of glycerin. These typically require either a high level (greater than 5% or even greater than 10%) of an emollient oil. such as triethylhexanoine, octyldodecyl myristate, or a high level (greater than 50%) of an alcohol. such as ethanol. The former (oil) is known to cause an oily feel that is a negative on the scalp and hair, and for the latter (alcohol), it is known that most consumers are afraid to apply to a scalp that is already sensitive due to dandruff symptoms. The present invention solubilizes such anti-dandruff actives by the combination of a surfactant (primarily needed for hair conditioning), and a specific acid (primarily needed to adjust pH). Nothing other than these two materials are required to formulate these anti-dandruff and scalp health actives into soluble form in a liquid.

Any of the anti-dandruff or other scalp actives may be in the composition in an amount from about 0.01% to about 3%, by weight of the composition. In the present invention, the anti-dandruff or other scalp actives may be from about 0.1 to 2%; may be from about 0.5 to 1% of antidandruff or other scalp actives.

### Non-Ionic Surfactant

The hair care composition of the present invention may comprise a nonionic surfactant. Nonionic surfactants are typically compounds produced by the condensation of alkylene oxide groups (hydrophilic in nature) with an organic hydrophobic compound, which can be aliphatic or alkyl aromatic in nature, but can include other surfactants that do not possess a charge group. In the present invention, the non-ionic surfactant may have the non-ionic surfactant have an HLB value of 10-15. Examples of classes of nonionic surfactants are:
a. Alkyl phenol ethoxylates. The polyethylene oxide condensates of alkyl phenols, e.g., the condensation products of alkyl phenols having an alkyl group containing from about 6 to 12 carbon atoms in either a straight chain or branched chain configuration, with ethylene oxide, the said ethylene oxide being present in amounts equal to 10 to 60 moles of ethylene oxide per mole of alkyl phenol. The alkyl substituent in such compounds can be derived from polymerized propylene, diisobutylene, octane, or nonane, for example.
b. Polyethylene glycol/polypropylene glycol block copolymers. Those derived from the condensation of ethylene oxide with the product resulting from the reaction of propylene oxide and ethylene diamine products which can be varied in composition depending upon the balance between the hydrophobic and hydrophilic elements which is desired. For example, compounds containing from about 40% to about 80% polyoxyethylene by weight and having a molecular weight of from about 5,000 to about 11,000 resulting from the reaction of ethylene oxide groups with a hydrophobic base constituted of the reaction product of ethylene diamine and excess propylene oxide, said base having a molecular weight of the order of 2,500 to 3,000, are satisfactory.
c. Fatty alcohol and fatty acid ethoxylates. The condensation product of aliphatic alcohols having from 8 to 18 carbon atoms, in either straight chain or branched chain configuration with ethylene oxide, e.g., a coconut alcohol ethylene oxide condensate having from 10 to 30 moles of ethylene oxide per mole of coconut alcohol, the coconut alcohol fraction having from 10 to 14 carbon atoms. Other ethylene oxide condensation products are ethoxylated fatty acid esters of polyhydric alcohols (e.g., Tween 20-polyoxyethylene (20) sorbitan monolaurate).
d. Long chain tertiary amine oxides. Long chain tertiary amine oxides corresponding to the following general formula:

R1R2R3N→O
wherein R1 contains an alkyl, alkenyl or monohydroxy alkyl radical of from about 8 to about 18 carbon atoms, from 0 to about 10 ethylene oxide moieties, and from 0 to 1 glyceryl moiety, and R2 and R3 contain from 1 to about 3 carbon atoms and from 0 to about 1 hydroxy group, e.g., methyl, ethyl, propyl, hydroxy ethyl, or hydroxy propyl radicals. The arrow in the formula is a conventional representation of a semipolar bond. Examples of amine oxides suitable for use in this invention include dimethyldodecylamine oxide, oleyldi(2-hydroxy ethyl) amine oxide, dimethyloctylamine oxide, dimethyldecylamine oxide, dimethyltetradecylamine oxide, 3,6,9-trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl) amine oxide, dimethylhexadecylamine oxide.
e. Long chain tertiary phosphine oxides. Long chain tertiary phosphine oxides corresponding to the following general formula:

RR′R″P→O
wherein R contains an alkyl, alkenyl or monohydroxyalkyl radical ranging from 8 to 18 carbon atoms in chain length, from 0 to about 10 ethylene oxide moieties and from 0 to 1 glyceryl moiety and R′and R″ are each alkyl or monohydroxyalkyl groups containing from 1 to 3 carbon atoms. The arrow in the formula is a conventional representation of a semipolar bond. Examples of suitable phosphine oxides are: dodecyldimethylphosphine oxide, tetradecylmethylethylphosphine oxide, 3,6,9-trioxaoctadecyldimethylphosphine oxide, cetyldimethylphosphine oxide, 3-dodecoxy-2-hydroxypropyldi(2-hydroxyethyl) phosphine oxide stearyldimethylphosphine oxide, cetylethylpropylphosphine oxide, oleyldiethylphosphine oxide, dodecyldiethylphosphine oxide, tetradecyldiethylphosphine oxide, dodecyldipropylphosphine oxide, dodecyldi(hydroxymethyl) phosphine oxide, dodecyldi(2-hydroxyethyl) phosphine oxide, tetra-decylmethyl-2-hydroxypropylphosphine oxide, oleyldimethylphosphine oxide, 2-hydroxydodecyldimethylphosphine oxide.
f. Long chain dialkyl sulfoxides. Long chain dialkyl sulfoxides containing one short chain alkyl or hydroxy alkyl radical of 1 to about 3 carbon atoms (usually methyl) and one long hydrophobic chain which contain alkyl, alkenyl, hydroxy alkyl, or keto alkyl radicals containing from about 8 to about 20 carbon atoms, from 0 to about 10 ethylene oxide moieties and from 0 to 1 glyceryl moiety. Examples include: octadecyl methyl sulfoxide, 2-ketotridecyl methyl sulfoxide, 3,6,9-trioxaoctadecyl 2-hydroxyethyl sulfoxide, dodecyl methyl sulfoxide, oleyl 3-hydroxypropyl sulfoxide, tetradecyl methyl sulfoxide, 3 methoxytridecyl methyl sulfoxide, 3-hydroxytridecyl methyl sulfoxide, 3-hydroxy-4-dodecoxy butyl methyl sulfoxide.
g. Alkyl polysaccharide (APS) surfactants such as the alkyl polyglycosides. Such surfactants are APS surfactants having a hydrophobic group with about 6 to about 30 carbon atoms and polysaccharide (e.g., polyglycoside) as the hydrophilic group. Optionally, there can be a polyalkylene-oxide group joining the hydrophobic and hydrophilic moieties. The alkyl group (i.e., the hydrophobic moiety) can be saturated or unsaturated, branched or unbranched, and unsubstituted or substituted (e.g., with hydroxy or cyclic rings).
h. Polyethylene glycol (PEG) glyceryl fatty esters, such as those of the formula R(O)OCH2 CH(OH)CH2 (OCH2 CH2) n OH wherein n is from about 5 to about 200, may be from about 20 to about 100, and R is an aliphatic hydrocarbyl having from about 8 to about 20 carbon atoms.

The compositions of the present invention may comprise Tween 80-polyoxyethylene(20) sorbitan monolaurate and may comprise from about 1% to about 15% of the non-ionic surfactant, may comprise 3 to 10% of a non-ionic surfactant, may comprise 5 to 10% of a non-ionic surfactant.

### Monocarboxylic and Polyprotic Acid

Monocarboxylic acid may be an α-hydroxy acid (AHA), a β-hydroxy acid (BHA), or a polyhydroxy acid (PHA). A BHA may be salicylic acid, which reduces or eliminates the need to add a preservative. In the present invention, the acid may be lactic acid and glycolic acid (both α-hydroxy acids) instead of salicylic acid. Polyhydroxy acids used may be lactobionic acid or gluconolactone. Other acids that may be used are ones having net −1 charge as their dominant species in the final pH of the composition, such as glutamic acid or aspartic acid, but likely require a preservative in the composition. Polyprotic acids such as maleic acid, oxalic acid, citric acid, EDTA, malic acid, tartaric acid, etc, and the compositions of the present invention may include them to adjust pH of the leave-on composition. In the present invention, combinations or mixtures of acids may be used. The present invention may comprise of polyprotic acid i.e. citric acid from about 0.02% to about 0.2%, may comprise from about 0.05 to about 0.1% of acid.

### Aqueous Carrier

The hair care composition of the present invention comprises an aqueous carrier. The level and species of the carrier are selected according to the compatibility with other components, and other desired characteristics of the product.

In the present invention, the aqueous carrier may be substantially water. Deionized water may be used. Water from natural sources including mineral cations can also be used, depending on the desired characteristic of the product. Generally, the compositions of the present invention comprise from about 20% to about 99%, may be from about 30% to about 95%, and may be from about 80% to about 95% water.

### Glycols

The hair care composition of the present invention may comprise solvents that may be dermatologically acceptable. In the present invention, the carrier may include water and solutions or mixtures of water with lower alkyl alcohols and/or polyhydric alcohols. Examples of lower alkyl alcohols are monohydric alcohols having 1 to 6 carbons such as ethanol, methanol, propanol, isopropanol, butanol, pentanol, and hexanol. Examples of polyhydric alcohols are glycols, such as dipropylene glycol, propylene glycol, butylene glycol, 1,4-butanediol, 3-allyloxy-1,2-propanediol, 1,2-hexanediol, 1,6-hexanediol, 1,3-butanediol, 1,3-propanediol, 2,2′-thiodiethanol, glycerin and other diols.

Other non-limiting examples of organic solvents include polyglycols such as polypropylene glycol, polyethylene glycol, mixture of butylene glycol, polypropylene glycol, polyethylene glycol and ethers, such as dipropylene glycol n-butyl ether, sugars, and sugar derivatives.

The compositions of the present invention may comprise propylene glycol and may comprise from about 1% to about 10% of the solvent, or from about 1% to about 5%, or may comprise from about 2 to about 3%.

### Water-Soluble Polymer

The hair care compositions of the present invention may comprise a water-soluble polymer. A hydrophobically modified polymer may be used while the overall concentration of the lamellar gel phase is low, in order to adjust viscosity of the composition. The hydrophobically-modified water-soluble polymer may have a natural origin backbone such as xanthum gum, cellulose, starch, guar, gellan, etc., plus combinations thereof, but is not limited to these. The water-soluble polymer may have a cationic charge. In the present invention the water-soluble polymer may be polyquaterniu-10, such as UCARE Extreme Polymer supplied by Dow Inc., or may be cetyl hydroxyethylcellulose, such as Polysurf 67 CS or Natrosol 330 PLUS CS, both supplied by Ashland. A polymer may be used that can help restore hydrophobicity of the hair surface, due to higher hydrophobicity of the polymer itself.

The compositions of the present invention may comprise from about 0.01% to about 0.5% of the water-soluble polymer, maybe from about 0.05 to about 0.25%.

### Scalp Health Actives

In the present invention, a scalp health active may be added to provide scalp benefits. This group of materials is varied and provides a wide range of benefits including anti-dandruff, anti-fungal, anti-microbial, moisturization, barrier improvement, and anti-oxidant, anti-itch, and sensates. Such skin health actives and scalp care actives include but are not limited to: zinc pyrithione, climbazole, selenium sulfide, vitamin E and F, salicylic acid, glycols, glycolic acid. PCA, PEGs, erythritol, glycerin, lactates, hyaluronates, allantoin and other ureas, betaines. sorbitol, glutamates, xylitols, menthol, menthyl lactate, isocyclomone, benzyl alcohol, and natural extracts/oils including peppermint, spearmint, argan, jojoba and aloe.

### Optional Ingredients

The compositions of the present invention can also additionally comprise any suitable optional ingredients as desired. For example, the composition can optionally include other active or inactive ingredients.

The compositions may include other common hair ingredients such as other anti-dandruff actives, minoxidil, conditioning agents, and other suitable materials. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of nonlimiting materials that can be added to the composition herein. Examples of these ingredient classes include, but are not limited to: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, niacinamide, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, rheology modifiers, hair conditioning agents, and surfactants.

### Acid

The hair care compositions of the present invention may comprise an acid. The acid may be used to control the pH of the composition to the desired range (3.5 to 6), because otherwise, the pH may be higher than 7 due to the presence of piroctone olamine.

### Fragrance

The compositions of the present invention may comprise a fragrance, or combination of fragrances. Some fragrances may comprise, multiple fragrance ingredients. In particular, the fragrance may comprise hinokitiol, phenoxyethanol, and L-menthol. The compositions may comprise the fragrance at the level solely required to design the desired fragrance character and strength, but not at a higher enough level that any of the materials act as a preservative for the composition.

### Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, may be up to about 5% by weight of the composition.

A wide variety of other additional components can be formulated into the present compositions. These include: other conditioning agents such as hydrolyzed collagen with tradename Peptein 2000 available from Hormel, vitamin E with tradename Emix-d available from Eisai, panthenol available from Roche, panthenyl ethyl ether available from Roche, hydrolyzed keratin, proteins, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; perfumes; and sequestering agents, such as disodium ethylenediamine tetra-acetate; and ultraviolet and infrared screening and absorbing agents such as octyl salicylate.

In the present invention, the compositions of the present invention may be free of preservatives. In the present invention, the compositions may be free of preservative compounds such as phenoxyethanol, benzyl alcohol, methyl paraben, ethyl paraben, and/or propyl paraben. While phenoxyethanol may be used as a fragrance at a level of at most 0.1%, by weight of the composition, it may not be used as a preservative, which would be at a level of at least 0.25%, by weight of the composition.

In the present invention the composition may be free of a liquid oily compound having an A log P≥5.5.

### Product Forms

The compositions of the present invention can be in the form of leave-on or rinse-off products and can be formulated in a wide variety of product forms, including but not limited to creams, gels, emulsions, mousses and sprays.

The composition of the present invention is especially suitable for hair leave-on. Such compositions may be used by following steps:
(i) after shampooing and/or after rinse-off conditioner hair, applying to the scalp and hair roots an effective amount of the compositions for scalp benefit; and
(ii) then not rinsing the hair or rinsing the hair.

### Translucence

The hair care compositions of the present invention is transparent, may be translucent. The translucence of the compositions may be consumer-preferred and can be a signal to apply to the scalp, leading to the anti-dandruff benefits.

The present invention compositions may have a viscosity from about 0.01 Pa·s to about 10 Pa·s, maybe from about 5 Pa·s to about 10 Pa·s. The lower viscosity compositions are more likely to be used on the scalp by consumers, thus more likely to result in anti-dandruff efficacy.

It may be consumer desirable to have the present invention composition with a minimal level of ingredients. The composition may be formulated without polymeric thickeners or suspending agents such as carbomer, EGDS or thixcin. The composition may be comprised of 11 or fewer ingredients, 10 or fewer ingredients, 9 or fewer ingredients, 8 or fewer ingredients, 7 or fewer ingredients, 6 or fewer ingredients. It is understood that perfumes can be formed from one or more materials.

### FORMULATIONS AND EXAMPLES

The following are non-limiting examples of the present invention. The examples are given solely for the purpose of illustration and are not to be construed as limitations of the present invention, as many variations thereof are possible without departing from the spirit and scope of the invention, which would be recognized by one of ordinary skill in the art.

### Formulation Preparation and Treatment Protocols:

### Leave-On Treatment Composition Preparation:

The leave-on treatment compositions are prepared by adding piroctone olamine with glycol and perfume, if needed, into non-ionic surfactant/water carrier and stirred until complete dissolution. The solution pH is adjusted using citric acid solution to a final pH of 4.0-6.0. The polymer (if required) is then added, if needed, and the solution is mixed using a high-speed-mixer for 2-5 minutes at 1800-2300 rpm until a uniform composition is obtained.

### Leave-On Hair Treatment Protocol:

An amount of 0.20 g of each composition of Examples I to IV is spread via a syringe onto separate natural virgin brown hair switches weighing 2.0 g (dosage 0.10 g of solution per g of hair). The hair is allowed to air dry and then it is assessed for sticky/coated feel by panel.

### Evaluation Methods

The hair switches that are treated with the leave on treatment compositions are evaluated using the following methodology.

Hair Switch Sticky Assessment Method: The treated hair switches are kept at room temperature, lab conditions (32-35° C., 50% RH) for 2 hrs and then ten expert graders are asked to rate each of them in terms of hair sticky feel based on a 5 point scale, 5 being the highest (very sticky) and I being the lowest rating (not sticky).

Phase stability: Sample is stored in glass jar at room temperature (32-35° C., 50% RH). Visible phase separation is recorded after 1 month. The same samples are observed by microscope with crossed-polarizer filter (Keyence Digital Microscope VHX-7000 series) for the presence of Piroctone Olamine crystal. S=Stable, U=Unstable.

Transparency: Transparency of sample is scored within a day of sample making. Transparency score is scored by panel for the formula, 1—Opaque, 3—Translucent and 5—Transparent.

### EXAMPLES

The following are non-limiting examples of the present invention. The following examples further describe and demonstrate examples within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

TABLE 1

| | | | | | Comparative | Comparative | Comparative | Comparative |
|---|---|---|---|---|---|---|---|---|
| | Ex. 1 | Ex. 2 | Ex. 3 | Ex. 4 | Ex. 5 | Ex. 6 | Ex. 7 | Ex. 8 |
| Deionized Water | Q.S. | Q.S. | Q.S. | Q.S. | Q.S. | Q.S. | Q.S. | Q.S. |
| Piroctone Olamine¹ | 0.30 | 0.30 | 0.30 | 0.30 | 0.30 | 0.30 | 0.30 | 0.3 |
| Propylene glycol² | 5.0 | 8.0 | 8.0 | 5.0 | 5.0 | 3.0 | 5.0 | 5.0 |
| Niacinamide³ | 2.5 | 2.5 | 2.5 | 2.5 | 2.5 | 2.5 | 2.5 | 2.5 |
| Tween-80⁴ | 10.0 | 10.0 | 10.0 | 10.0 | 10.0 | 10.0 | 0.0 | 10.0 |
| Mosa Mint Oil⁵ | 0.50 | 0.5 | 0.0 | 0.3 | 0.5 | 0.5 | 0.5 | 0.5 |
| Panthenol⁶ | 0.25 | 1.0 | 0.25 | 0.25 | 0.25 | 0.25 | 0.25 | 0.25 |
| Citric acid⁷ | 0.1 | 0.02 | 0.1 | 0.1 | 0.05 | 0.1 | 0.1 | 0.1 |
| Benzyl Alcohol⁸ | 0.25 | 0.25 | 0.25 | 0.25 | 0.25 | 0.25 | 0.25 | 0.25 |
| Xanthum gum⁹ | 0.0 | 0.0 | 0.0 | 0.0 | 0.0 | 0.0 | 0.0 | 0.2 |
| Fragrance Base | 0.00 | 0.0 | 0.5 | 0.0 | 0.0 | 0.0 | 0.00 | 0.00 |
| Phase stability | S | S | S | S | U | U | U | S |
| pH | 5.5 | 5.7 | 5.7 | 5.8 | 7.2 | 5.8 | 5.75 | 5.5 |
| Sticky Feel on Hair | 2 | 3 | 2 | 2 | 2 | 1.0 | 3 | 2 |
| Switch (graded on scale | | | | | | | | |
| of 5, 5 being very | | | | | | | | |
| sticky and 1 not sticky) | | | | | | | | |
| Transparent/translucent/ | Transparent | Transparent | Transparent | Transparent | Transparent | Transparent | Transparent | Translucent |
| Opaque | | | | | | | | |
| (5-Transparent, 3- | | | | | | | | |
| Translucent, 1-Opaque) | | | | | | | | |

¹Octopirox, Clariant
²Dow
³Merck
⁴Tween80, Croda
⁵Labbeemint, Inc
⁶Sigma Aldrich
⁷Jungbunzlauer Austria
⁸Supplied by Ineos Maastricht BV (Maastricht NL)
⁹Cargill

Results: Formulation Ex. 1 to 4 show transparent, stable and less sticky formula with pH 5.5-5.8 whereas Ex. 5 is unstable due to high pH crystal formation as demonstrated after 1 day of formulation making. Ex. 6 and Ex. 7 are unstable due to less of solvent glycol and absence of Tween 80 to form microemulsion respectively. Ex 8. is translucent with addition of Xanthum gum vs Ex. 1 to 4 transparent.

### Combinations

A. A transparent hair care composition comprising a substituted 2-pyridinol-N-oxide material; a glycol; a non-ionic surfactant; a monocarboxylic acid or polyprotic acid; water or an aqueous carrier; wherein the composition has a pH from about 3.5 to about 6.0; and wherein the composition is free of alcohol.
B. A transparent hair care composition according to Paragraph A, wherein the non-ionic surfactant has an HLB value of 10-15.
C. A transparent hair care compositions according to Paragraph A-B, comprising from about 1% to about 10% of glycol selected from the group consisting of dipropylene glycol, propylene glycol, butylene glycol, 1,4-butanediol, 3-allyloxy-1,2-propanediol, 1,2-hexanediol, 1,6-hexanediol, 1,3-butanediol, 1,3-propanediol, 2,2′-thiodiethanol or glycerin.
D. A transparent hair care composition according to Paragraph A-C, wherein the glycol is propylene glycol.
E. A transparent hair care composition according to Paragraph A-D, comprising from about 1% to about 15% of the non-ionic surfactant selected from the group consisting of alkyl phenol ethoxy lates, Polyethylene glycol/polypropylene glycol block copolymers, fatty alcohol and fatty acid ethoxylates, long chain tertiary amine oxides, long chain tertiary phosphine oxides, long chain dialkyl sulfoxides, alkyl polysaccharide or polyethylene glycol glyceryl fatty esters and mixtures thereof.
F. A transparent hair care composition according to Paragraph A-E, wherein the non-ionic surfactant is Tween 80-polyoxyethylene (20) sorbitan monolaurate.
G. A transparent hair care composition according to Paragraph A-F, comprising from about 0.02% to about 0.2% of monocarboxylic acid selected from the group consisting of a α-hydroxy acid (AHA), a β-hydroxy acid (BHA), or a polyhydroxy acid (PHA) and mixtures thereof.
H. A transparent hair care composition according to Paragraph A-G, wherein the polyprotic acid is selected from the group consisting of maleic acid, oxalic acid, citric acid, EDTA, malic acid or tartaric acid and mixtures thereof.
I. A transparent hair care composition according to Paragraph A-H, wherein the polyprotic acid is citric acid.
J. A transparent hair care composition according to Paragraph A-I, further comprising from about 0.01% to about 0.5% of a water-soluble polymer.
K. A transparent hair care composition according to Paragraph A-J, wherein the water-soluble polymer has a natural origin backbone selected from the group consisting of cellulose, starch, guar, or gellan and mixtures thereof.
L. A transparent hair care composition according to Paragraph A-K, wherein the water-soluble polymer is selected from the group consisting of polyquaterniu-10 or cetyl hydroxyethylcellulose and mixtures thereof.
M. A transparent hair care composition according to Paragraph A-L, further comprising a scalp health active.
N. A transparent hair care composition according to Paragraph A-M, wherein the composition is non-sticky and stable.
O. A transparent hair care composition according to Paragraph A-N, wherein the composition is free of an anionic surfactant.
P. A transparent hair care composition according to Paragraph A-O, wherein the composition consists of 9 or fewer ingredients.
Q. A transparent hair care composition according to Paragraph A-P, consisting of
   a. water;
   b. piroctone olamine;
   c. propylene glycol
   d. niacinamide;
   e. Tween-80;
   f. mosa mint oil;
   g. panthenol;
   h. citric acid; and
   i. benzyl alcohol.
R. A transparent hair care composition according to Paragraph A-Q, consisting of
   a. water;
   b. piroctone olamine;
   c. propylene glycol
   d. niacinamide;
   e. Tween-80;
   f. mosa mint oil;
   g. panthenol;
   h. citric acid;
   i. benzyl alcohol; and
   j. optionally, a perfume.

It will be appreciated that other modifications of the present disclosure are within the skill of those in the hair care formulation art can be undertaken without departing from the spirit and scope of this invention. All parts, percentages, and ratios herein are by weight unless otherwise specified. Some components may come from suppliers as dilute solutions. The levels given reflect the weight percent of the active material, unless otherwise specified. A level of perfume and/or preservatives may also be included in the following examples.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A transparent hair care composition comprising:
a) a substituted 2-pyridinol-N-oxide material;
b) a glycol;
c) a non-ionic surfactant;
d) a monocarboxylic acid or polyprotic acid;
e) water or an aqueous carrier;
wherein the composition has a pH from about 3.5 to about 6.0; and wherein the composition is free of alcohol.

2. A transparent hair care composition according to claim 1 wherein the non-ionic surfactant has an HLB value of 10-15.

3. A transparent hair care composition according to claim 1 comprising from about 1% to about 10% of glycol selected from the group consisting of dipropylene glycol, propylene glycol, butylene glycol, 1,4-butanediol, 3-allyloxy-1,2-propanediol, 1,2-hexanediol, 1,6-hexanediol, 1,3-butanediol, 1,3-propanediol, 2,2′-thiodiethanol or glycerin.

4. A transparent hair care composition according to claim 3 wherein the glycol is propylene glycol.

5. A transparent hair care composition according to claim 1 comprising from about 1% to about 15% of the non-ionic surfactant selected from the group consisting of alkyl phenol ethoxylates, Polyethylene glycol/polypropylene glycol block copolymers, fatty alcohol and fatty acid ethoxylates, long chain tertiary amine oxides, long chain tertiary phosphine oxides, long chain dialkyl sulfoxides, alkyl polysaccharide or polyethylene glycol glyceryl fatty esters and mixtures thereof.

6. A transparent hair care composition according to claim 5 wherein the non-ionic surfactant is Tween 80-polyoxyethylene (20) sorbitan monolaurate.

7. A transparent hair care composition according to claim 1 comprising from about 0.02% to about 0.2% of monocarboxylic acid selected from the group consisting of a α-hydroxy acid (AHA), a β-hydroxy acid (BHA), or a polyhydroxy acid (PHA) and mixtures thereof.

8. A transparent hair care composition according to claim 1 wherein the polyprotic acid is selected from the group consisting of maleic acid, oxalic acid, citric acid, EDTA, malic acid or tartaric acid and mixtures thereof.

9. A transparent hair care composition according to claim 1 wherein the polyprotic acid is citric acid.

10. A transparent hair care composition according to claim 1 further comprising from about 0.01% to about 0.5% of a water-soluble polymer.

11. A transparent hair care composition according to claim 10 wherein the water-soluble polymer has a natural origin backbone selected from the group consisting of cellulose, starch, guar, or gellan and mixtures thereof.

12. A transparent hair care composition according to claim 11 wherein the water-soluble polymer is selected from the group consisting of polyquaterniu-10 or cetyl hydroxyethylcellulose and mixtures thereof.

13. A transparent hair care composition according to claim 1 further comprising a scalp health active.

14. A transparent hair care composition according to claim 1 wherein the composition is non-sticky and stable.

15. A transparent hair care composition according to claim 1 wherein the composition is free of an anionic surfactant.

16. A transparent hair care composition according to claim 1, wherein the composition consists of 9 or fewer ingredients.

17. A transparent hair care composition, consisting of:
a. water;
b. piroctone olamine;
c. propylene glycol
d. niacinamide;
e. Tween-80;
f. mosa mint oil;
g. panthenol;
h. citric acid; and
i. benzyl alcohol.

18. A transparent hair care composition, consisting of:
a. water;
b. piroctone olamine;
c. propylene glycol
d. niacinamide;
e. Tween-80);
f. mosa mint oil;
g. panthenol;
h. citric acid;
i. benzyl alcohol and
j. optionally, a perfume.
